(19) United States
(12) Patent Application Publication
(10) Pub. No.: US 2022/0415437 A1
(43) Pub. Date: Dec. 29, 2022

(54) COVER SET DETERMINATION FOR IDENTIFYING BIOLOGICAL ENTITIES

(71) Applicant: International Business Machines Corporation, Armonk, NY (US)

(72) Inventors: Mark Kunitomi, San Francisco, CA (US); Samyukta Satish Rao, San Jose, CA (US); Daniel Waddington, Morgan Hill, CA (US); Amir Abboud, Sunnyvale, CA (US)

(21) Appl. No.: 17/356,614

(22) Filed: Jun. 24, 2021

(51) Int. Cl. G16B 20/40

(52) U.S. Cl. CPC G16B 20/40

(57) ABSTRACT

A computer-implemented method for generating a cover set of biological sequences to detect a group of biological members. The method includes one or more computer processors receiving a request to generate a cover set of k-mers used to detect a first group of biological members that are related via a taxonomic lineage. The method further includes obtaining a plurality of biological sequence data corresponding to biological members of the first group. The method further includes determining a set of k-mers respectively associated with a biological member included within the first group of biological members. The method further includes determining the cover set of k-mers utilized to detect the biological members of the first group by selecting a subset of k-mers from a superset of k-mers associated with the first group of biological members based on preventing false-positive detections of biological members different from the first group of biological members.

### BACKGROUND OF THE INVENTION

The present invention relates generally to the field of bioinformatics, and more particularly to determining sets of biological sequences to identify related biological entities.

The genetic diversity among bacteria, viruses, and other microorganisms has led to their inhabitance of almost every known habitable niche on earth. However, various microorganisms, pathogens, and organic substances generated by microorganisms represent one of the greatest threats to public and food safety. Rapid assessment of the proper treatment for an infection caused by a pathogen has drastic effects on patient outcome. For example, patients with typhoid fever that do not receive timely and appropriate treatment are estimated to have a 30% mortality rate, whereas that mortality rate from typhoid fever can be reduced to just 0.5% for patients that receive timely and appropriate treatment.

Nucleic acid-based detection systems, such as the Polymerase Chain Reaction (PCR), are the primary class of rapid diagnostic tools to determine the identity of various pathogens, such as bacteria and viruses. PCR tests have a wide range of applications, including detecting pathogens in food ingredients and products, characterizing environmental microbiota, and diagnosing infectious diseases. The success of an identification test depends on the ability of the assays within the test to identify sequences (i.e., signatures) that properly differentiate between the target organism(s) and the sample background, which includes all other organisms potentially present in a sample.

### SUMMARY

According to an aspect of the present invention, there is a computer-implemented method, computer program product, and/or system for generating a cover set of biological sequences to detect a group of biological members. In an embodiment, the method includes at least one computer processor receiving a request to generate a cover set of k-mers used to detect a first group of biological members that are related via a taxonomic lineage. The method further includes at least one computer processor obtaining a plurality of biological sequence data corresponding to biological members of the first group. The method further includes at least one computer processor determining a set of k-mers respectively associated with a biological member included within the first group of biological members. The method further includes at least one computer processor determining the cover set of k-mers utilized to detect the biological members of the first group by selecting a subset of k-mers from a superset of k-mers associated with the first group of biological members based on preventing false-positive detections of biological members different from the first group of biological members.

### BRIEF DESCRIPTION OF THE DRAWINGS

FIG. 1 illustrates a networked-computing environment, in accordance with an embodiment of the present invention.

FIG. 2 depicts a flowchart of steps of a biological analysis program, in accordance with an embodiment of the present invention.

FIG. 3 depicts a flowchart of steps of a member grouping program, in accordance with an embodiment of the present invention.

FIG. 4 depicts a flowchart of steps of cover set generation program, in accordance with an embodiment of the present invention.

FIG. 5 is a block diagram of components of a computer, in accordance with an embodiment of the present invention.

### DETAILED DESCRIPTION

Embodiments of the present invention recognize that the widespread genetic and related trait diversities among microorganisms cause difficulties in the ability to detect, categorize, and combat microorganisms. Embodiments of the present invention also recognize that with decreased costs and increases in accessibility of high-throughput nucleic acid sequencing systems that the number of genomes of microorganisms that have been sequenced and cataloged has increased rapidly. The massive amount of genomic (e.g., nucleic acid) sequence data (as of November 2017, GenBank alone stored >100,000 genomes) presents an opportunity to capture the diversity of microorganisms, but also presents a challenge of developing methods that can computationally derive signatures associated with microorganisms and related biological features, such as proteins, phenotype-based traits, etc. In addition, embodiments of the present invention recognize that rapid sequencing and identification of biological materials (e.g., microorganisms, proteins) are crucial to public safety; detecting and preserving beneficial microorganism; identifying novel or dormant microorganism in previously undisturbed locals; and detecting the effects of human activities on various microorganisms, such as an increase of genetic transfers and/or mutations, or changes to the biodiversity of an area.

Embodiments of the present invention recognize that the ability to determine characterizing features and/or nucleic acid sequences of populations, bacterial isolates, sub-species, and serovars/serotypes in the context of the diversity of microorganisms is also useful with respect to food safety and clean water. In one example, the salmonella genus of bacteria includes over 2600 serotypes. In another example, Vibrio cholerae, the species of bacteria that causes cholera, has over 200 serotypes, based on cell antigens. However, only two serotypes have been observed to produce the potent enterotoxin that results in sever cholera.

Embodiments of the present invention recognize that a major limitation of nucleic acid-based detection systems is that specific a priori information (e.g., nucleic acid sequences) about target sequences and off-target sequences is necessary in order to generate specificity of a method. Further, the a priori information must also include information related to the diversity of the larger population of microorganisms within the environment.

Embodiments of the present invention further recognize that due to sharing of genetic material that exists via vertical descent; horizontal gene transfers, such as plasmid transfer and viral remnants; and convergent evolution that, in many cases, there is no single sequence of nucleic acids of a targetable length (i.e., a signature) that meets the required sensitivity and specificity criteria to identify different groups of microorganisms. For example, no single signature is found within all of the desired target genomes for a group of microorganisms.

Embodiments of the present invention utilize the concept of cover sets to identify related biological entities. Embodiments of the present invention improve the speed for determining the minimal number of genomic signatures (e.g., k-mers and/or contigs) that in combination describe a cover set of target genomes or other biological sequences.

Embodiments of the present invention utilize that fact that genomic signatures of imperfect sensitivities can be identified in order to complement each other and in combination, achieve the required sensitivity and specificity to identify groups of microorganisms and other related biological entities, such as ribonucleic acid (RNA), messenger RNA, transcriptions, etc., that are linear polymers based on a sequence of organic compounds (i.e., monomers) of a known alphabet. For example, terrestrial genes are polynucleotide chains are composed of the nucleotides: adenine, cytosine, guanine, and thymine respectively denoted by A, C, G, and T. Whereas, RNA is composed of nucleotides adenine, cytosine, guanine, and uracil [U].

Similarly, some embodiments of the present invention are adapted and applied to detecting and identifying other biological substances, such as a subset of proteins that are linear polymers based on a sequence of other organic compounds (i.e., amino acids) of another known alphabet. For example, “alphabet” of proteins is based on 20 standard amino acids utilized to create most proteins. Other embodiments of the present invention can also reconstruct contiguous sequences of biological data corresponding to deoxyribonucleic acid (DNA), RNA, and/or proteins with greater in silico determined sensitivity and specificity with respect to a user-defined criteria.

Various embodiments of the present invention are further utilized to specify the elements included within an assay array to produce physical tests that can identify biological members within a sample based on a dictated sensitivity and specificity scores. By determining the minimal number of k-mers, contigs, or other genomic signatures to identify organisms, biological members, or other biological substances, embodiments of the present invention reduce the cost testing in addition to reducing user error. Another embodiment of the present invention can perform the detection and identification in silico by directly analyzing sequence data obtained from a sample against databases of known sequence data corresponding to a plurality of biological members or other biological materials. Thus, generating a practical application.

Other embodiments of the present invention enable the determination and inclusion of additional k-mer based assay elements within a physical test to improve to detection of previously unknown (e.g., outlier) variants of a biological member or group of biological members, detect new genetic traits (i.e., phenotype-based) incorporated within a known biological member, and/or detect one or more mutations within a known biological member.

Further embodiments of the present invention utilized the results of one or more tests and/or identifications to interface with various health, safety, and regulatory databases to determine one or more responses based on the one or more biological members that are identified within a sample and the source of the sample, such as a soil sample, a fecal sample, a sample from a raw foodstuff, samples along a foodstuff supply chain, a biopsy, etc.

In one embodiment, the terms “sequence” or “biological sequence” refers to a DNA or RNA nucleotide sequence and/or an amino acid sequence of proteins. Within the context of embodiments of the present invention, biological sequences include genes, contigs, sequences and sub-sequences from any genome, the latter including, without limitation, genomes of multi-cellular organisms (e.g., humans, plants, animals), prokaryotic genomes, eukaryotic genomes, genomes of viruses, and other biological entities, such as tumor cells, cancer cells.

In an embodiment, the term “metadata” refers to the descriptions and sampling sites and habitats that provide the context for sequence information. Examples of metadata include, without limitation, geographical location of the sample, a date, features of the environment of the sample, chemical data from the sample, method of sampling, sample size, sample preparation.

In one embodiment, the term “member grouping” used herein refers to a group based on a taxonomic rank, such as genus selected from one or more of a genome, gene, protein, domain, and/or other sequence of biological information. In some embodiments, the term “member sequence” or “member sequences” refers to one or more sequences that comprise a particular genus. For example, within each genus-based member grouping will be individual species with one or more sequences specific to those species; and may further include sub-species, variants, and serovars of a particular genus. Further, a “biological member” can refer to a particular microorganism, biological material, cell type, etc.

As used herein, the term “signature” and “biological signature” refers to one or more biological sequences (e.g., nucleic acid-based or amino acid-based) that differentiate an individual species or other biological member from a sample background (i.e., the one or more member sequences of the background).

As used herein, the term “contig” refers to a set of overlapping sequences that represent a contiguous sequence from a sequence assembly, the latter being known in the art as a sequence that is reconstructed from the aligning and merging of DNA fragments and/or k-mers from a longer DNA, RNA, or protein sequence.

As used herein, the term “k-mer” refers to an individual from a set of all the possible overlapping substrings of length k that are contained in a string or set of strings. In bioinformatics, the term k-mer refer to all of the possible overlapping sub-sequences of length k contained within a biological sequence. Within the context of computational genomics and sequence analysis, k-mers are composed of nucleotides A, C, T, G, U, and N (any nucleotide, or an ambiguous nucleotide) or amino acids (e.g., the 20 amino acids that make up proteins). A sequence of length L will have L−k+1 k-mers and n^{k} total possible k-mers, where n is the number of possible monomers (e.g., four nucleotides in the case of DNA or RNA and 20 amino acids in the case of proteins). Using nucleotides as an example, the sequence AGAT has four monomers (A, G, A, T), three 2-mers (AG, GA, AT), and one 4-mer (AGAT).

The descriptions of the various scenarios, instances, and examples related to the present invention have been presented for purposes of illustration but are not intended to be exhaustive or limited to the embodiments disclosed.

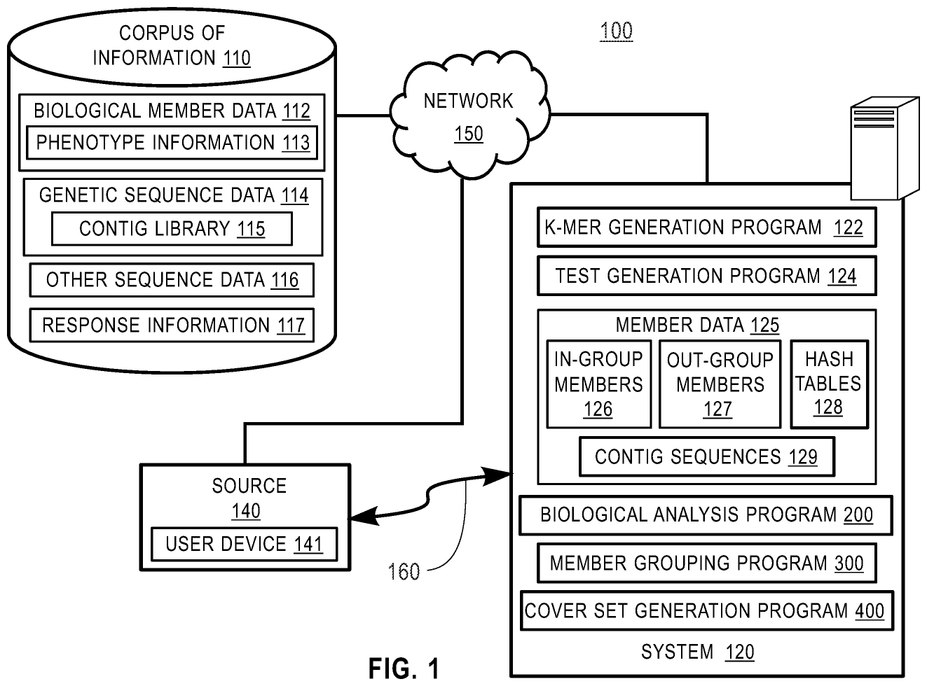

The present invention will now be described in detail with reference to the Figures. FIG. 1 is a functional block diagram illustrating networked-computing environment 100, in accordance with embodiments of the present invention. In an embodiment, networked-computing environment 100 includes corpus of information 110, system 120, and user device 141, all interconnected over network 150. In addition, networked-computing environment 100 also includes transfer path 160. In one embodiment, transfer path 160 represents a physical method (e.g., mechanism) to transport biological material and/or samples (not shown) from source 140 to system 120; or to supply requested tests kits to source 140 from system 120. In another embodiment, a different instance of transfer path 160 represents a physical method to supply requested test kits to source 140 from another entity (not shown) that fabricates and/or stores an inventory of test kits for owners/administrators of system 120 based on at least cover set data. As used herein, tests and test kits may be used interchangeably.

In an embodiment, corpus of information 110 represents a plurality of data, information, and a priori knowledge accessible from among public, private, institutional, medical, and/or government databases and electronic libraries. Corpus of information 110 includes biological member data 112, genetic sequence data 114, other sequence data 116, and response information 117. In an embodiment, one or more portions of corpus of information 110 are updated based on information determined by one or more aspects of system 120.

In an embodiment, biological member data 112 includes a plurality of taxonomic information related to microorganisms and other biological materials, such as taxonomic ID (TaxID) values or other unique identifiers, metadata corresponding to biological members, and other descriptive information. In various embodiments, biological member data 112 is cross-referenced, linked, and/or mapped to other information and data within corpus of information 110, such phenotype information 113, genetic sequence data 114, and response information 117. In some embodiments, biological member data 112 includes more detailed information included within phenotype information 113 and/or genetic sequence data 114. For example, biological member data 112 may also include information that identifies different genetic-based diseases that can affect a biological member, such as cancers or metabolic disorders.

Phenotype information 113 includes information associated with traits or features that have a genetic basis, such as therapeutic resistances (e.g., drug and/or radiation resistances), resistances to environmental factors, sensitivities to environmental factors, therapeutic sensitivities, chemical sensitivities, longevity on various surfaces, genetic diseases can affect a biological member, etc. Environmental factors may include temperature, pH, salinity, pressure, types of light, etc. In some embodiments, information within phenotype information 113 is cross-referenced or mapped among a plurality of various biological members; and is further linked or mapped to data included within genetic sequence data 114 and other sequence data 116.

In an embodiment, other sequence data 116 includes data related to the sequences of other linear polymers, such as proteins that are based on an alphabet of monomers. In some embodiments, an element of other sequence data 116 is associated with a sequence within genetic sequence data 114 that produces a given linear polymer.

In an embodiment, response information 117 includes a plurality of dictates, regulations, treatment regimes, and other data and information related to responding to the identification of one or more biological materials and/or phenotype-based traits among biological sequences obtained from a sample or detected utilizing a k-mer based test. In one example, response information 117 may dictate notifying governmental authorities when the detected microorganism is variant that includes additional genetic material identified within phenotype information 113 that may confer a new trait that is flagged for concern, such as a drug resistance. In another example, response information 117 indicates that a foodstuff at one stage of processing must be destroyed if any member of genus of a given microorganism is identified (i.e., detected) by a test. Response information 117 can also include alternative responses based on one or more criteria or conditions, such as saving a foodstuff from destruction at another stage of processing if the foodstuff can be processed in a manner that eliminates the identified microorganism based on also detecting one or more sensitivities (e.g., traits) included within biological member data 112 and/or phenotype information 113.

Corpus of information 110, system 120, and user device 141 may be laptop computers, tablet computers, netbook computers, personal computers, desktop computers, personal digital assistants (PDA), smart phones, wearable devices (e.g., smart glasses, a smart watch, an e-textile, an AR headsets, etc.), or any programmable computer systems known in the art. In certain embodiments, corpus of information 110, system 120, and user device 141 represent computer systems utilizing clustered computers and components (e.g., database server computers, application server computers, etc.) that act as a single pool of seamless resources when accessed through network 150, as is common in data centers and with cloud-computing applications.

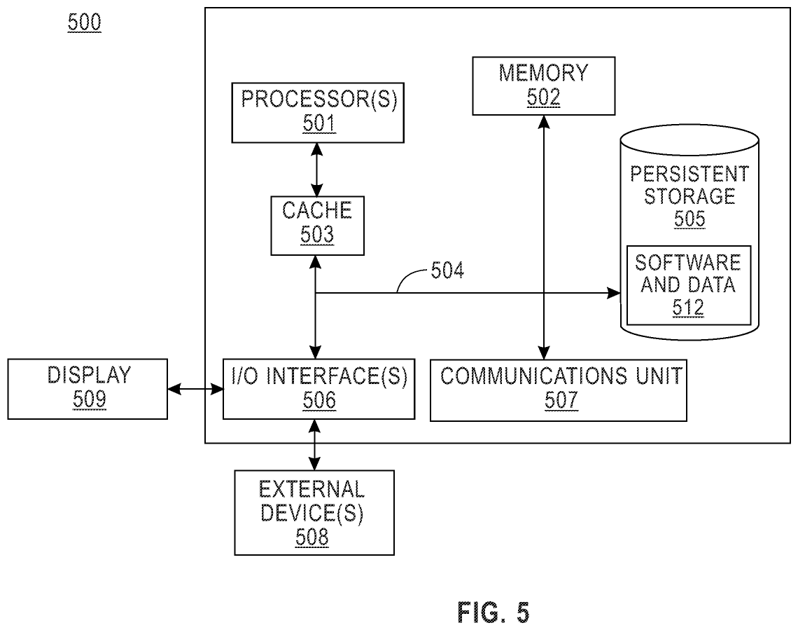

In general, corpus of information 110, system 120, and user device 141 are representative of any programmable electronic device or combination of programmable electronic devices capable of executing machine readable program instructions and communicating via network 150 with corpus of information 110, system 120, and user device 141. Corpus of information 110, system 120, and user device 141 may include components, as depicted and described in further detail with respect to FIG. 5, in accordance with embodiments of the present invention.

System 120 includes k-mer generation program 122, test generation program 124, member data 125, biological analysis program 200, member grouping program 300, cover set generation program 400, and other programs and data (not shown). Examples of other programs and data include an operating system (OS), a query generation program, a database management system, one or more communication programs, etc. In some embodiments, system 120 also includes and/or interfaces with a plurality of other features or hardware elements (not shown), such as a genetic sequencer, a biological assay/test kit fabrication system, etc.

K-mer generation program 122 is a program that generates a set of k-mers that correspond to a biological member or another biological sequence related to a biological member. K-mer generation program 122 can invoke various file and database functions, such as unzip a file, append data and metadata to a database, etc. K-mer generation program 122 also includes a plurality of formulas and algorithms that perform various of mathematical operations, logical operations, set operations, and interpret set notations. Examples of set notations, functions, and symbols, include Ø (empty set), [symbol] (universal set), ∈ (an element of . . . ), ∩ (intersection), U (union), etc. In addition, K-mer generation program 122 parallelizes the analysis of genomes across threads (i.e., each genome is processed by exactly one thread) to ensure that k-mer frequency counts (i.e., occurrences) are only incremented once per genome and are maintained in thread-local storage (TLS).

K-mer generation program 122 also converts a plurality of alphabetical nucleotide sequences, contigs, genes or protein sequences into a numeric format to improve the in silico processing of information. In an embodiment, if a biological member is sequenced and stored as a FASTA file, then k-mer generation program 122 unzips the FASTA file corresponding to the biological member and converts each contig to 3-bit raw (bit packed) format. For example, a genome g of a biological member is given as:
an unordered set of ‘contigs’ g={c₀, c₁, c₂, . . . cₙ}; and
a contig is an ordered sequence of bases (b) such that:

c₁=(b₀,b₁,b₂, . . . bₘ):b∈{A,C,T,G,N}.

In one embodiment, k-mer generation program 122 encodes each nucleotide base (b) of a contig utilizing a binary format, such as A=001, C=010, G=100, T=111, N=011, and U=110. The aforementioned encoding method utilized by k-mer generation program 122 maintains alphabetical ordering of a contig with respect to the numerical ordering of the contig. K-mer generation program 122 also writes each contig to log-storage (append only) within contig sequences 129 and adds metadata related to a contig, such as a filename, record index, and FASTA metadata to biological member information 121 (e.g., a database). In addition, K-mer generation program 122 also applies exclusive OR (XOR) 0x7 to the binary value corresponding to each base b to derive the complement b′ (b prime) value corresponding to each base b.

In another embodiment, k-mer generation program 122 further determines a given k-mer k, which is a (sliding window) substring within contig c of length L (nominally 100 bases), that is calculated according to Formula (1) and the set of k-mers corresponding to contig c determined by Formula (2).

$${k}_{i}^{L} = canonical\_choice ( ( {b}_{i} , {b}_{i + 1} , {b}_{i + 2} , … {b}_{i + L - 1} ) , ( {b}_{i + L - 1}^{′} , {b}_{i + L - 2}^{′} , … {{b}_{i}}^{′} ) ) ; \\ where i \geq 0 and i < \vert c \vert - L \qquad ( 1 )$$

$${K}^{L} ( c ) = {{⋃}^{\vert c \vert - L}}_{i = 0} {k}_{i}^{L} \qquad ( 2 )$$

Another aspect of k-mer generation program 122 determines a hash value corresponding to a given k-mer k utilizing a hash functions and stores the hash value within a hash table of hash tables 128 based on an identifier corresponding to the biological member that includes the sequence that includes the identified k-mer. Utilizing hash values corresponding to k-mers enables more rapid set operation execution and frequency of occurrence determinations for a given k-mer among a plurality of biological members. For example, k-mer generation program 122 loads the 3-bit nucleotide data and converts the 3-bit data into an in-memory 8-bit form. The 8-bit form enables performing a sliding window extraction. In addition, k-mer generation program 122 generates a 64-bit hash of the converted 8-bit-per-base DNA string of nucleotide data corresponding to an identified k-mer. K-mer generation program 122 performs sliding window extraction on all contigs for all genomes of the biological members utilized during an analysis. K-mer generation program 122 increments k-mer occurrences only once for each new genome. In addition, k-mer generation program 122 determines a canonical form for each identified k-mer (see Formula (1)). K-mer generation program 122 may utilize a dual-sliding window approach to avoid reversals at a cost of transient memory footprint.

In response to determining a cover set of k-mers, a different aspect of k-mer generation program 122 reverses the binary encoding of the set of k-mers and outputs the determined cover set of k-mers as sequences of nucleotides (i.e., A, C, T, G, U) or amino acids.

Test generation program 124 is a program that utilizes criteria received in a request from a user associated the source 140 and information generated by at least biological analysis program 200 to generate and/or order a test utilizing an array of k-mer (e.g., sequences of nucleotides or sequences of amino acids) based assays to identify a biological member, a group of biological members, and/or one or more phenotype-based traits. In one embodiment, test generation program 124 determines an array of assays detect a target biological member or a group of target biological members to a dictated sensitivity score (e.g., percentage of group members detected) and a dictated specificity score. In some embodiments, test generation program 124 interfaces with corpus of information 110 to identify k-mer based phenotype information to further include within a test based on a request from a user. Alternatively, test generation program 124 can utilize a cover set of k-mer data based on a relative compliment of k-mers determined by cover set generation program 400 to determine one or more additional assays used to identify a dictated phenotype-based trait or related genetic sequence.

Member data 125 includes In-group members 126, Out-group members 127, hash tables 128, and contig sequences 129. In various embodiments, member data 125 also includes a plurality of sequence data and related metadata corresponding to one or more biological members within a sample or included within a request from a user. In an embodiment, member data 125 further includes at least the ID's of a universal set (U) of reference genomes used in analyses. Member data 125 includes a plurality of origin vectors for one or more groups and/or the results of a set operation. Member data 125 may also include cross-references between unique member IDs (e.g., specific ID's or designations utilized by a particular program and/or algorithm) and one or more TaxIDs based on a taxonomic level or ranking. In a further embodiment, member data 125 includes data determined by cover set generation program 400 based on dictates included within the request received by biological analysis program 200, such as a different universal set based on a relative compliment (not shown) associated with one or more phenotype-based traits to include within a test.

In some embodiments, some sequence data and information is download from corpus of information 110 to member data 125 and/or contig sequences 129 on a temporary basis. Members can be genomes, genes, proteins, domains, or other sequences of biological information. In-group (I) refers to the group of biological members to identify. Out-group (O) refers to the biological members to be excluded from being mis-identified as members of the In-group. Where: I_{G}={g:g∈ [symbol] }; O_{G}={g:g∈ [symbol] }; and I_{G}∩O_{G}=Ø.

In an embodiment, In-group members 126 includes at least a TaxID or other unique identifier corresponding to each biological member that member grouping program 300 assigns to an In-group associated with a request. In-group members are the group biological members that are specified within a request to be identified. Biological members within In-group members 126 may be further associated with a ranked taxonomic sub-division. In various embodiments, biological members within In-group members 126 are mapped to information included within a hash table of hash tables 128 and/or member data 125, such as origin vectors.

In an embodiment, Out-group members 127 includes at least a TaxID or other unique identifier corresponding to each biological member that member grouping program 300 assigns to an Out-group associated with a request. Out-group members are the group biological members to excluded from identification, which may be somewhat similar to the In-group. For example, if an In-group is one genus of anerobic bacteria, then another genus of anerobic bacteria may be assigned to an Out-group. However, a genus of aerobic bacteria could be assigned to a null set/group (not shown). Biological members within Out-group members 127 may be further associated with a ranked taxonomic sub-division. In various embodiments, biological members within Out-group members 127 are mapped to information included within a hash table of hash tables 128.

Hash tables 128 includes a plurality of hash tables respectively associated with a group (e.g., an In-group or an Out-group) associated with a request and includes key-value pairs respectively associated with a biological member and further associated with a k-mer included within one or more sequences related to the biological member. In an embodiment, hash tables 128 includes one or more hash tables based on at least key-values pairs of: key=a hash value corresponding to k-mer k, and value=a number of occurrences of k-mer k within a genome or other biological sequence. In some embodiments, hash tables 128 further information or hash tables respectively associated with a ranked sub-group of a group, such as Out-group 2. In various embodiments, hash tables 128 is also associated with the origin vectors related to at least k-mers of In-group members.

Contig sequences 129 includes a plurality of contig sequences of biological members. In one embodiment, contig sequences 129 includes information downloaded from corpus of information 110. In another embodiment, contig sequences 129 includes sequences k-mer data related to one or more biological members associated with a physical sample or included within a request from a user. In some embodiments, contig sequences 129 includes one or more different contigs generated during the execution of cover set generation program 400.

Biological analysis program 200 is a program for identifying biological materials utilizing k-mer based cover set information determined utilizing k-mer generation program 122, member grouping program 300, and cover set generation program 400. In embodiment, biological analysis program 200 identifies one or more biological members in silico based on data-to-data comparisons and a set of criteria defined within a request by a user. In another embodiment, biological analysis program 200 also utilizes test generation program 124 to determine one or more sets of k-mer based assays to include within a physical test that can identify one or more biological members, or a group of biological members based on criteria specified by a user within a request. In one scenario, biological analysis program 200 instructs system 120 to produce or supply one or more tests. In one scenario, biological analysis program 200 instructs another entity (not shown) to produce or supply one or more tests.

In various embodiments, biological analysis program 200 also determines a set of responses based an identification of one or more biological members or results of a test (e.g., biological member/phenotype detection), and/or a location/situation where the sample was obtained or the test was performed; and communicates the responses to at least a user that submitted the identification request and/or another user associated with source 140.

Member grouping program 300 is a program that assigns a plurality of biological members among an In-group and an Out-group based on a query generated in response to a request from a user to identify a biological member or group of biological members. Member grouping program 300 utilize information included within the request, such as a TaxID or other unique identifier of a group of biological members, an intersection TaxID, a sensitivity score and specificity criteria (e.g., a score, a level, a rank). In some scenarios, a specificity criterion is a taxonomy rank as opposed to a calculated value or score. In other scenarios, the specificity criteria are the ranked sub-divisions assigned to at least Out-group members. Member grouping program 300 also utilizes k-mer generation program 122 and biological sequence data corresponding to a biological member within corpus of information 110 and/or received sequence data to determine a set of k-mers corresponding to each biological member within In-group members 126 and out-group members 127.

In one embodiment, member grouping program 300 accesses corpus of information 110 to determine the known TaxIDs corresponding to lineage(s) of biological members included within ranked TaxID specified within the request. In some embodiments, member grouping program 300 further assigns biological member within a group among ranked taxonomic sub-divisions within the group (e.g., Out-group 1, 2, . . . ). In various embodiments, member grouping program 300 also determines corresponding k-mer data for a member ID and stores related data among hash tables 128, such as a k-mer hash value and an occurrence value of a k-mer among biological member IDs, where the biological member ID can differ from a TaxID. Member grouping program 300 can determine a superset of k-mers among biological members of a group.

Cover set generation program 400 determines the set of k-mers and/or contigs (i.e., the cover set) that are required to describe and detect the members of a specified group of biological members. Cover set generation program 400 utilizes various mathematical and set operations to determine the k-mers and/or contigs that are identified within In-group members but do not occur among Out-group members based on a dictated sensitivity score and a specificity score or criteria. In various embodiments, cover set generation program 400 iteratively generates the cover set of k-mers based on one or more dictates included within a request, such as starting the cover set based on elements (e.g., k-mers or contigs) found among the smallest quantity of biological members of the In-group. Cover set generation program 400 utilizes information included within hash tables 128 to determine the frequency of occurrences of k-mers among biological members. Cover set generation program 400 also utilizes the plurality of origin vectors within member data 125 for various determinations and calculations. In some instances, cover set generation program 400 may determine a cover set consisting of a smaller number of large contigs as opposed to a combination of k-mers and contigs. In a further embodiment, cover set generation program 400 generates multiple (e.g., a dictated number) cover sets based on an intentional redundancy dictate included within the received request.

In an embodiment, source 140 represents a location (stationary or mobile) where biological samples are obtained and/or checked with one or more test kits. In another embodiment, source 140 includes one or more systems (not shown) that prepare and sequences biological material and samples. In various embodiments, source 140 also includes at least one computing device, such as user device 141 that communicate with at least system 120 via network 150.

In some embodiments, user device 141 associated with source 140 is utilized by a user to submit identification requests/queries, send sequence data, transmit results corresponding to a k-mer based test, and/or receive results/responses related to identifying a biological material. For example, a user utilizes user device 141 to specify the requirements of one or more test kits that system 120 supplies to source 140 via transfer path 160. In response, a user associated with source 140 analyzes one or more samples utilizing the supplied test kits (not shown) and utilizes user device 141 to transmit the results of the test to system 120. In response, user device 141 may receive a set of responses from system 120 based on results obtained by testing a sample.

Network 150 can be, for example, a local area network (LAN), a telecommunications network (e.g., a portion of a cellular network), a wireless local area network (WLAN), such as an intranet, a wide area network (WAN), such as the Internet, or any combination of the previous and can include wired, wireless, or fiber optic connections. In general, network 150 can be any combination of connections and protocols that will support communications between corpus of information 110, system 120, and user device 141, in accordance with embodiments of the present invention. In various embodiments, network 150 operates locally via wired, wireless, or optical connections and can be any combination of connections and protocols (e.g., personal area network (PAN), near field communication (NFC), laser, infrared, ultrasonic, etc.).

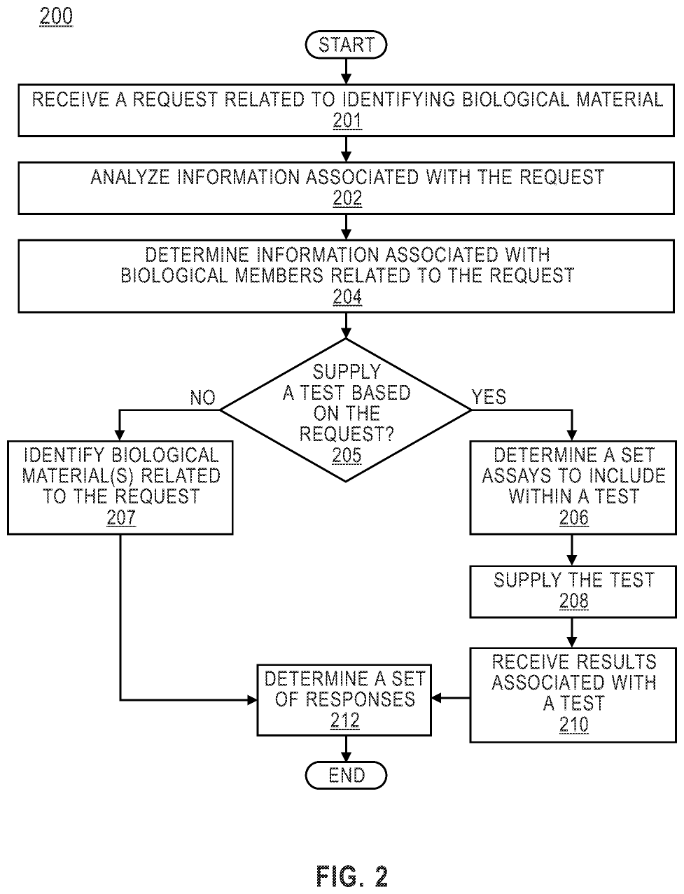

FIG. 2 is a flowchart depicting operational steps for biological analysis program 200, a program for identifying biological materials utilizing based on analyzing a plurality of biological sequence information, biological sequence data corresponding to a sample, and various dictates associated with a user request, in accordance with embodiments of the present invention. In addition, biological analysis program 200 also determines a set of responses based on identifying one or more biological materials/members and further based on the source and/or location of the biological material(s). In various embodiments, biological analysis program 200 utilizes k-mer generation program 122, member grouping program 300, and/or cover set generation program 400 for various analyses and determinations.

In step 201, biological analysis program 200 receives a request related to identifying biological material. In one embodiment, biological analysis program 200 receives a request to identify biological material from a user associated with source 140. The request may include plurality of information, dictates, and/or criteria, such a TaxID of a target genus to identify, biological intersection criteria, a sensitivity score and a specificity score or criterion (e.g., rank); an indication designating that a data-to-data analysis is performed; a physical test is supplied for use at a source 140; a description of source 140 (e.g., a hospital, a natural location, an industrial plant). In some embodiments, biological analysis program 200 also receives sequence data from source 140. In other embodiments, biological analysis program 200 determines that a physical sample is received from source 140 via transfer path 160. Subsequently, biological analysis program 200 has the physical sample sequenced by another feature of system 120 or another entity (not shown) and stores the data within contig sequences 129.

In step 202, biological analysis program 200 analyzes information associated with the request. In an embodiment, biological analysis program 200 analyzes information associated with the request to determine criteria used for selecting biological member data and sequences obtained from corpus of information 110 utilize by k-mer generation program 122, member grouping program 300, and/or cover set generation program 400. For example, biological analysis program 200 analyzes information associated with the request to determine the superset of biological members to select from during various analyses, the biological members to targeted for identification, the biological members or groups of members to exclude from a cover set, etc.

In a further embodiment, biological analysis program 200 analyzes the received request to determine additional dictates or criteria related to identifying or detecting phenotype-based trait information, such as sensitivities and/or resistances, telltale metabolic by-products, etc. In one example, if the biological member identified by biological analysis program 200 is a water-borne pathogen, then biological analysis program 200 also utilizes corpus of information 110 to determine the number and frequency of testing. In another example, biological analysis program 200 determines that the received request includes dictates and risk criteria related to saving or recycling an organic material (e.g., a foodstuff), if that organic material is contaminated with one or more biological members associated with the request.

In step 204, biological analysis program 200 determines information associated with the biological members related to the request. In an embodiment, biological analysis program 200 utilizes corpus of information 110 to determine the TaxID corresponding to biological members associated with the request, lineage information corresponding to a biological member, and members of a given lineage to a dictated level of specificity. In some embodiments, biological analysis program imports genetic sequence data and/or other sequence data for known biological members from corpus of information 110 for subsequent parsing and analysis utilizing aspects of k-mer generation program 122. In another embodiment, biological analysis program 200 determines that the received request includes a dictate to generate a cover set based on intentional redundancy criteria as opposed to a minimum cover set of k-mers.

In various embodiments, biological analysis program 200 executes k-mer generation program 122, member grouping program 300, and cover set generation program 400 to determine information associated with the biological members related to the request, such as a determining a cover set used in an analysis, or a cover set used to determine a set of assays to include within a test, additional k-mers or contigs to include on a test improve the sensitivity or specificity of a test, additional k-mers and/or contigs used to detect dictated phenotype-based traits, etc. For example, biological analysis program 200 imports additional sequence data from corpus of information 110 corresponding to a phenotype-based trait included within the request. In a further embodiment, if biological analysis program 200 determines data and information is not included within corpus of information 110, then biological analysis program 200 uploads the new data and information to corpus of information 110 for subsequent curation.

In decision step 205, biological analysis program 200 determines whether to supply a test based on the request. In one scenario, biological analysis program 200 determines to supply a test based on information included in the request. In another scenario, biological analysis program 200 determines to perform data-to-data analyses as opposed to suppling a test based on information included in the request. In some scenarios, biological analysis program 200 determines, based on information received in request to both perform data-to-data analyses and to supply or produce one or more tests for subsequent samplings at source 140.

Responsive to determining not to supply a test based on the request (No branch, decision step 205), biological analysis program 200 identifies biological material(s) related to the request (step 207).

In step 207, biological analysis program 200 identifies the biological material(s). In an embodiment, biological analysis program 200 identifies one or more biological materials related to the received request by performing various in silico data-to-data analyses based on one or more sequences of data included within the received request and a cover set determined based on various criteria and dictates included within the received request. In another embodiment, biological analysis program 200 identifies one or more biological materials related to the received request by performing various in silico data-to-data analyses based on one or more genomic sequences derived or isolated from a sample received from source 140, and a cover set determined based on various criteria and dictates included within the received request. In some embodiments, biological analysis program 200 identifies other information based the received sequence data and other dictates or criteria included within the received request, such as identifying an undocumented serovar of a bacterium or flagging the detection of a horizontal gene transfer between biological members. Subsequently, biological analysis program 200 determines a set of responses based on the identified biological material(s) (in step 212).

Referring to decision step 205, responsive to determining to supply a test based on the request (Yes branch, decision step 205), biological analysis program 200 determines a set of assays to include within a test (step 206).

In step 206, biological analysis program 200 determines a set of k-mer based assays to include within a test. In some embodiment, biological analysis program 200 utilizes test generation program 124 to determine a set of k-mer based assays (e.g., analyte elements, target sequences) to include within a test based on the cover set determined in response to the received request. In other embodiments, biological analysis program 200 utilizes test generation program 124 to includes one or more additional assay elements based on information included within the received request, such as additional k-mer assays to improve the sensitivity and/or specificity of the test, lower specificity assay element to improve the detection of outliers (e.g., previously unknown biological members) for a group of biological members, additional assay elements to detect one or more dictated phenotype-based traits, additional assay elements to further identify one or more biological members of particular concern (e.g., more virulent sub-species), etc.

In another embodiment, in response to determining that the request included intentional redundancy criteria, biological analysis program 200 interfaces with a user to select or modify the redundancies to include within a cover set before proceeding. For example, biological analysis program 200 may present a user with a graphical or pictorial representation of multiple cover sets of k-mers and/or contigs depicting a frequency distribution, heat map, or overlap map among the biological entities to identify including a minimum k-mer cover set as reference. In response to receiving a user selection of k-mers, and/or other dictates, biological analysis program 200 can re-execute one or more steps cover set generation program 400. If the user indicates that the cover set determination is complete, then biological analysis program 200 determines assays to include within a test.

In step 208, biological analysis program 200 supplies a test. In various embodiments, one or more tests are supplied to a user associated with source 140 via transfer path 160 from an entity that produces assay tests and/or stores an inventory of common tests. In one embodiment, if system 120 includes the capabilities to produce biological assay tests based on k-mer information, then biological analysis program 200 instructs system 120 to produce (e.g., fabricate) one or more tests, such as microarrays based on at least the determined cover set and the received request. In another embodiment, biological analysis program 200 contacts another entity (not shown) to produce one or more tests based on at least the determined cover set and the received request and deliver the tests (not shown) to source 140. As is known in the art, tests include other components, such as reagents and other materials to enable the test in addition to a plurality of assays utilized to detect target sequences (e.g., designated k-mers and/or contigs).

In step 210, biological analysis program 200 receives results associated with a test. In an embodiment, in response to a user utilizing one or more test on biological materials (e.g., exposing a test to a sample from source 140) within source 140, biological analysis program 200 receives results associated with one or more tests. For example, results of one or more tests may be input to user device 141 by a user interpreting visual indicators within a test, utilizing user device 141 to send an image of a microarray test, or upload digital results of an electronic microarray and transmitted to system 120 via network 150.

In step 212, biological analysis program 200 determines a set of responses. In various embodiments, biological analysis program 200 utilizes information within corpus of information 110 to determine a set of responses based on identifying one or more biological materials, via in silico analyses or the results of one or more physical tests (e.g., results of an array of assays). In one example, biological analysis program 200 determines a set of responses that include treatment regimens based on identifying a species of pathogen and narrowing the treatment regimens to a specific treatment based on identifying a particular sub-species of the pathogen. In another example, biological analysis program 200 determines a set of responses that dictate dispositioning a material (e.g., destroying, purifying, modifying a preparation process, recalling a distributed material) associated with source 140 based on the use of the material, risk factors, and/or regulatory requirements. In addition, biological analysis program 200 may determine that the set of responses also includes notification requirements, such as a supplier notifications, customer notifications, notification of one or more regulatory agencies/departments.

In some embodiments, the nature and/or location of source 140 affects one or more responses determined by biological analysis program 200. In one example, if test results analyzed by biological analysis program 200 identifying a particular micro-organism within materials of a wholesale distribution center triggers tracing and inspections throughout a supply chain back to one or more sources of a raw material. In another example, biological analysis program 200 may update corpus of information 110 in response to identifying a biological member within a location or organism where the biological member did not previously occur. In another embodiment, biological analysis program 200 determines other responses in response to determining that the received request includes additional dictates, criteria, or a particular identifier related to identifying phenotype-based trait information, such as sensitivities and/or resistances; detecting horizontal gene transfers among biological members, or detecting new variants/mutations within a population of biological members of concern.

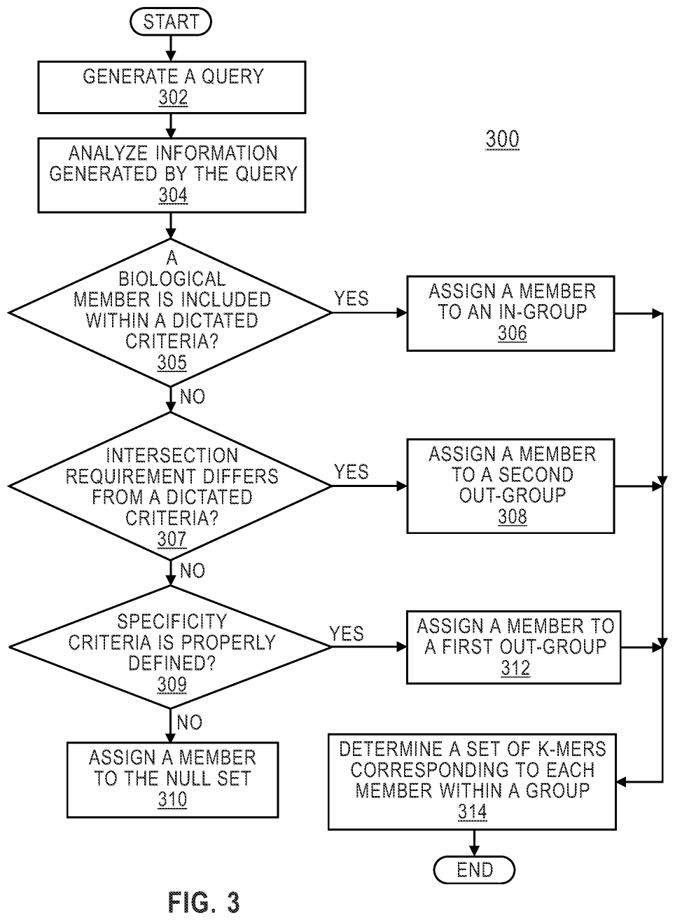

FIG. 3 is a flowchart depicting operational steps for member grouping program 300, a program that assigns a plurality biological members identified by a query among an In-group and at least one Out-group based on taxonomic information and criteria defined within a request from a user, in accordance with embodiments of the present invention. In an embodiment, member grouping program 300 can assign biological members to other groups, such as null set. In various embodiments, member grouping program 300 forwards to grouping results to cover set generation program 400 for further analyses.

In step 302, member grouping program 300 generates a query. In an embodiment, member grouping program 300 generates a query associated with biological members based on information within the request received by biological analysis program 200. For example, member grouping program 300 generates a query that dictates the criteria of TaxID of a species of biological members to identify, one or more sets/supersets of biological members to include (e.g., potential background organisms to distinguish from, unrelated contamination) within an analysis, a TaxID related to a biological intersection, a specificity value and/or taxonomic rank, etc. Member grouping program 300 may access corpus of information 110 to obtain sequence data related to other biological members to include within a query that differ from the biological sequence data associated with the request received by biological analysis program 200.

In various embodiments, member grouping program 300 also accesses corpus of information 110 to determine the taxonomic lineage for each biological member identified or associated with a dictate within the request from a given level of specificity to a lower of specificity, such as Family. In a further embodiment, member grouping program 300 also includes other information in the query specified within the received request. For example, member grouping program 300 may also obtain phenotype data and corresponding sequence data from corpus of information 110 in addition to TaxIDs or other unique identifiers corresponding to other biological member that include the specified phenotype data.

In step 304, member grouping program 300 analyzes information generated by the query. In addition, member grouping program 300 parses the request received by biological analysis program 200 to identify various dictates, criteria, and/or values utilized in various determinations and decisions. Member grouping program 300 may access corpus of information to determine data and information related to one or more dictates or criteria, such as identifying the member of a taxonomic lineage. In one embodiment, member grouping program 300 analyzes a portion of the information generated by the query to determines a plurality of lineages of biological members and corresponding TaxIDs or other related identifiers utilized for various comparisons or decisions.

In some embodiments, member grouping program 300 determines information corresponding to biological members to exclude from one or more determinations or groupings. In various embodiments, member grouping program 300 analyzes each biological member identified by the query based on one or more criteria and determinizations until all the identified biological members are assigned to an In-group, an Out-group, or the null set (not shown).

In decision step 305, member grouping program 300 determines whether a biological member is included within a dictated criteria. In one embodiment, member grouping program 300 determines that a biological member is included within a first criterion dictated within the received request, such as a TaxID of a species, a TaxID of a sub-species, a specified lineage, a member of a family of proteins, includes a dictated phenotype-based trait, or generates a dictated biological compound. For example, if member grouping program 300 determines that the query TaxID of a biological member is a part of a dictated the lineage from a higher specificity rank (e.g., strain as opposed to species), then member grouping program 300 assigns the biological member to In-group members 126. In a further embodiment, member grouping program 300 determines that a biological member is assigned In-group member 126 based on two or more criteria dictated within the received request.

Responsive to determining that a biological member is included within a dictated criteria within the received request (Yes branch, decision step 305), member grouping program 300 assigns a member to an In-group (step 306).

In step 306, member grouping program 300 assigns a member an In-group. In an embodiment, member grouping program 300 assigns the biological member, based on the dictated criteria, to In-group members 126 and updates member data 125 and/or a respective hash table within hash tables 128 with the TaxID or other unique identifier corresponding the biological member. In a further embodiment, member grouping program 300 can also include the IDs corresponding to each phenotype-based trait dictated within the received request for inclusion as a member of In-group member 126.

Referring to decision step 305, responsive to determining that a biological member is not included within a dictated criteria within the received request (No branch, decision step 305), member grouping program 300 determines to determine whether intersection requirements differs from a dictated criteria (decision step 307).

In decision step 307, member grouping program 300 determines whether intersection requirements differs from a dictated criteria. (decision step 307). In one embodiment, member grouping program 300 determines whether an intersection requirement for a biological member differs from a second dictated criterion, such as an in-lineage intersection TaxID. For example, if member grouping program 300 determines that a biological member is not included within a lineage of biological entities at or below a dictated ranked TaxID respectively associated with an intersection criteria, then member grouping program 300 determines that the intersection criteria for the biological member differs. Alternatively, if member grouping program 300 determines that a biological member is included within a lineage of biological entities at or below a dictated ranked TaxID, then member grouping program 300 determines that the intersection criteria for the biological member does not differ.

Responsive to determining that the intersection requirement differs from a dictated criteria (Yes branch, decision step 307), member grouping program 300 assigns a member to a second Out-group (step 308).

In step 308, member grouping program 300 assigns a member to a second Out-group. In an embodiment, member grouping program 300 assigns one or more the biological members, to a second group of Out-group members included withing Out-group members 127 and updates member data 125 and/or a respective hash table within hash tables 128 with the TaxID or other unique identifier corresponding the included biological members.

Referring to decision step 307, responsive to determining that the intersection requirement for a biological member does not differ from a dictated criteria (No branch, decision step 307), member grouping program 300 determines to determine whether the specificity criteria is properly defined (decision step 309).

In decision step 309, member grouping program 300 determines to determine whether the specificity criteria is properly defined. In one embodiment, member grouping program 300 determines that the specificity criteria is properly defined based on determining that a biological member is included within a lineage but occurs within a different taxonomic rank. In an embodiment, member grouping program 300 determines that the specificity criteria is not properly defined based on determining that a biological member is included within an unrelated lineage. For example, anerobic bacteria as opposed to aerobic bacteria, bacteria as opposed to viruses, etc. In another embodiment, member grouping program 300 determines that the specificity criteria for is not properly defined based on determining that one or more portions of the query was too broad.

Responsive to determining that the specificity criteria is not properly defined (No branch decision step 309), member grouping program 300 assigns a member to the null set (step 310).

In step 310, member grouping program 300 assigns a member to the null set (step 310). Member grouping program 300 excludes members assigned to the null set from further analyses.

Referring to decision step 309, responsive to determining that the specificity criteria is properly defined (Yes branch decision step 309), member grouping program 300 assigns a member to a first Out-group (step 312).

In step 312, member grouping program 300 assigns a member to a first Out-group. In an embodiment, member grouping program 300 assigns the member, based on the dictated specificity, to Out-group members 127 and updates member data 125 and/or a respective hash table within hash tables 128 with the TaxID or other unique identifier corresponding the biological member.

In step 314, member grouping program 300 determines a set of k-mers corresponding to each member within a group. In an embodiment, member grouping program 300 utilizes k-mer generation program 122 to determine a set of k-mers corresponding to each biological member among In-group members 126 and Out-group members 127. In addition, member grouping program 300 generates a respective hash tables and/or creates hash table entries within hash tables 128 for In-group members 126 and Out-group members 127. In various embodiments, member grouping program 300 creates respective hash tables based on key-values pairs where: key=a hash value corresponding to k-mer k (previously discussed with respect to k-mer generation program 122), and value=a number of occurrences of k-mer k within a genome or other biological sequence. Another key-value pairing specifies value as the number of members of In-group member 126 that include a particular k-mer k.

In addition, member grouping program 300 also generates respective origin vectors for each identified k-mer of the plurality of biological members within In-group members 126 and stores the origin vectors within member data 125. An origin vector consists of all the IDs (e.g., TaxIDs or other unique identifiers) of biological members that include one or more occurrences of a given k-mer. Member grouping program 300 also performs various set operations and frequency-of-occurrence determinations (discussed below) associated with k-mers with respect to the members of In-group members 126 and Out-group members 127.

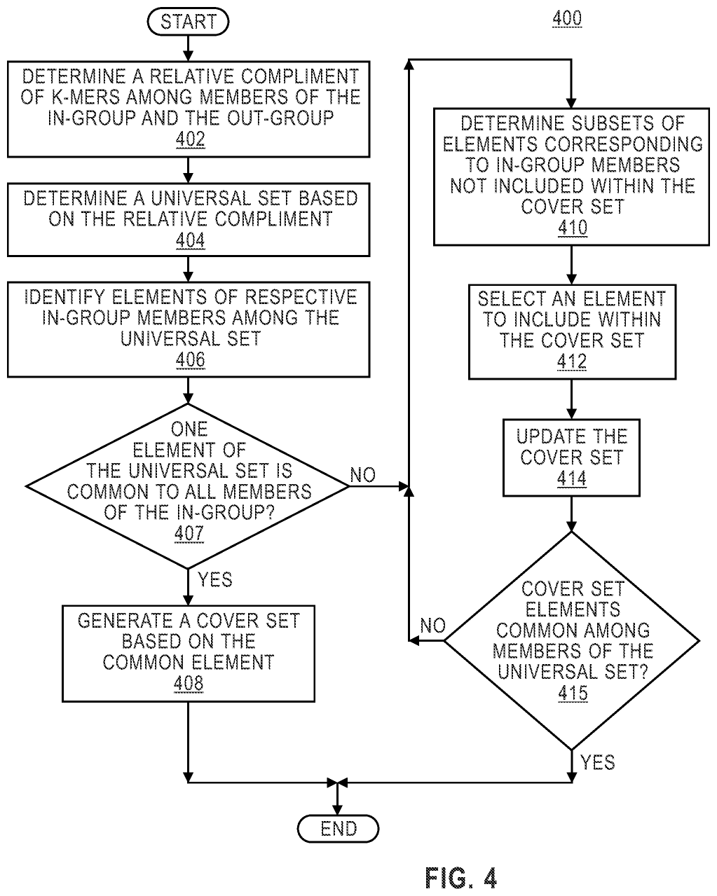

FIG. 4 is a flowchart depicting operational steps for cover set generation program 400, a program for determining a cover set of k-mers utilized to identify a group of microorganisms or other biological materials to a dictated specificity value/criterion, in accordance with embodiments of the present invention.

In step 402, cover set generation program 400 determines a relative complement of k-mers among members of the In-group and the Out-group. In one embodiment, cover set generation program 400 determines a relative compliment with respect to In-group members 126 by removing all k-mers from an In-group set of k-mers that also occur in the Out-group set (e.g., k-mer occurrence value >0). For example, cover set generation program 400 iterates over each k-mer hash value within the hash table (not shown) included within hash tables 128 that corresponds to Out-group members 127, to identify identical k-mer hash values within a different hash table (not shown) included within hast tables 128 that corresponds to In-group members 126 to determine a value for the number of occurrences of a given k-mer of among In-group members 126 with respect to Out-group members 127 (e.g., determining the relative complement between supersets of k-mers). In a further embodiment, cover set generation program 400 determines an additional relative compliment based on the k-mers corresponding to one or more phenotype-based traits dictated within the received request.

In another embodiment, cover set generation program 400 determine a relative compliment of k-mers based on an epsilon value (i.e., a threshold number of occurrences) dictated within the received request and/or the query. For example, cover set generation program 400 determines the relative compliment based on a set of k-mers with a sensitivity score equal to the percentage of members within the In-group member 126 that contain a given k-mer as calculated by dividing number of true positives by the sum of true positive and false negatives (TP/(TP+FN)) and specificity score equal to the percentage of members, outside of In-group members 126 that does not contain the k-mer as calculated by dividing true negatives by the sum of true negatives plus false positives (TN/(TN+FP)). While the occurrence count of a given k-mer is less than the epsilon value, cover set generation program 400 continues to scan through the In-group hash table for occurrences of a give k-mer from the Out-group hash table. In addition, cover set generation program 400 may periodically scrub the In-group hash table of k-mers in response to determining that frequency count for one or more k-mers is greater than the dictated epsilon value. In other embodiments, a different relative complement is determined based on specificity value or criterion corresponding to a taxonomic rank.

Still referring to step 402, in addition, in some embodiments cover set generation program 400 also filters the relative compliment based on user-defined thresholds corresponding the sensitivity and specificity values of respective k-mers. Subsequently, cover set generation program 400 examines the post-filtered relative compliment of k-mers to determine whether two or more k-mers can be joined to create a longer contig. Each contig will consist of unique k-mers, such that created (i.e., joined) contigs will not overlap by more than K−1 bases. Contigs end when there is a branch or dead-end in the k-mer graph. Cover set generation program 400 temporally stores the created contigs within contig sequences 129.

In step 404, cover set generation program 400 determines a universal set based on the relative compliment. Cover set generation program 400 stores the universal set (not shown) of k-mers within member data 125 and also creates an empty cover set (not shown) within member data 125. In an embodiment, cover set generation program 400 performs a union operation of all the TaxIDs or other unique identifiers included within the origin vectors in each of the k-mers or contigs (created in step 402) that remain after determining the relative compliment to determine the universal set k-mers associated with the biological members to identify. In a further embodiment, cover set generation program 400 determines an additional set of one or more k-mers based on the relative compliment derived from one or more phenotype-based dictates included in the received request. Cover set generation program 400 may also store the additional set of k-mers separately within member data 125 as a basis of additional assay elements.

In step 406, cover set generation program 400 identifies elements of respective In-group members among the universal set. In an embodiment, cover set generation program 400 identifies and counts the quantity of biological members within In-group members 126 that include an element (e.g., a k-mer or a contig of joined k-mers) of the universal set of k-mers.

In decision step 407, cover set generation program 400 determine whether one element of the universal set is common to all members of the In-group. In some embodiments, cover set generation program 400 determine whether one element of the universal set is common to all members of the In-group In another embodiment, cover set generation program 400 determine that more than one element of the universal set is common to all member of the biological members of In-group members 126.

Responsive to determining that at least one element of the universal set is common to all members of the biological members within In-group members 126 (Yes branch, decision step 407), cover set generation program 400 generates a cover set based on the common element (step 408).

In step 408, cover set generation program 400 generates a cover set based on the common element, such as a contig. In some embodiments, cover set generation program 400 cover set generation program 400 generates a cover set based on determining that at least two elements common among all In-group members 126 occur within the universal set. Cover set generation program 400 stores the determined cover set within member data 125. In another embodiment, cover set generation program 400 executes multiple times to determine differing cover sets based on an intentional redundancy dictate included within the received request. Cover set generation program 400 generates multiple cover sets by utilizing different filters; contig creations; minimum/maximum k-mer coverage as a starting point; random selections of uncovered elements; and/or one or more specific dictates by the user, such as specifying one or more k-mers or contigs as sequences to include within or exclude from each different cover set.

Referring to decision step 407, responsive to determining that one element of the universal set is not common to all members of the In-group (No branch, decision step 407), cover set generation program 400 determines subsets of elements corresponding to In-group members not included within the cover set (step 410).

In step 410, cover set generation program 400 determines subsets of elements corresponding to In-group members not included within the cover set. In one embodiment, cover set generation program 400 determines subsets of elements corresponding to biological members within In-group members 126 that are not included within the empty cover set based on comparing the origin vectors remaining after determining the relatively compliment to elements of the universal set. The TaxID or unique ID of a biological member included within In-group members 126 may occur within more than one subset. In another embodiment, cover set generation program 400 deletes (e.g., excludes) one or more determined subsets of elements corresponding to biological members within In-group members 126 based on updating the cover set with one or more elements of the universal set.

In step 412, cover set generation program 400 selects an element to include within the cover set. In one embodiment, cover set generation program 400 selects an element to include within the cover set from among the elements of the universal set that are not represented (i.e., included) within the cover set. In various embodiments, cover set generation program 400 iterates through the cover set and the universal set of k-mers to identify elements of the universal set (i.e., k-mers or contigs) not included within the cover set for subsequent element selections. In one scenario, cover set generation program 400 selects an element to include within the cover set based on identifying one or more subsets that include that largest quantity of biological members of In-group members 126 that are not covered by an element of the cover set. For example, cover set generation program 400 may review the origin vectors and select an element of the universal set based on the origin vectors that includes the largest of quantities of members.

In another scenario, cover set generation program 400 selects an element to include within the cover set based on identifying one or more subsets that includes that include elements unique to singular biological members of In-group members 126 that are not covered by an element of the cover set. For example, cover set generation program 400 may determine that there are two joined contigs that occur only once, in two different respective biological members among the plurality of biological members within In-group members 126. In response, cover set generation program 400 selects the two respective members. In a different scenario, cover set generation program 400 identifies two or more elements of the universal set of k-mers that are unique to two or more respective groups of biological members of In-group members 126. In response, cover set generation program 400 may select the two or more elements to add to the cover set.

In step 414, cover set generation program 400 updates the cover set. In an embodiment, cover set generation program 400 update the cover set stored within member data 125 with the one or more selected elements of the universal set that were not previously included within the cover set.

In decision step 415, cover set generation program 400 determines whether cover set elements common among the members of the universal set. In one embodiment, cover set generation program 400 determines that each member or element of the universal set is also included within the cover set. In another embodiment, cover set generation program 400 determines that at least one element of the universal set is not included within the cover set.

Responsive to determining that cover set elements common among the members of the universal set are not within (e.g., represented within) the cover set, (No branch, decision step 415), cover set generation program 400 loops to step 410 to determine subsets of elements corresponding to In-group members not included within the updated cover set. In some embodiments, cover set generation program 400 loops and skips to step 412 based on various criteria, such as identifying and selecting unique elements, or elements found within biological members of a given sensitivity, such as k-mers with zero Out-group occurrences.

Referring to decision step 415, responsive to that cover set elements common among the members of the universal set are within the cover set, (Yes branch, decision step 415), cover set generation program 400 returns the results of the cover set to biological analysis program 200 in step 204. In some embodiments, cover set generation program 400 also informs biological analysis program 200 that different cover of k-mer related to phenotype-based dictates are also determined and stored within member data 125.

FIG. 5 depicts computer system 500, which is representative of corpus of information 110, system 120, and user device 141. Computer system 500 is an example of a system that includes software and data 512. Computer system 500 includes processor(s) 501, cache 503, memory 502, persistent storage 505, communications unit 507, input/output (I/O) interface(s) 506, and communications fabric 504. Communications fabric 504 provides communications between cache 503, memory 502, persistent storage 505, communications unit 507, and input/output (I/O) interface(s) 506. Communications fabric 504 can be implemented with any architecture designed for passing data and/or control information between processors (such as microprocessors, communications and network processors, etc.), system memory, peripheral devices, and any other hardware components within a system. For example, communications fabric 504 can be implemented with one or more buses or a crossbar switch.

Memory 502 and persistent storage 505 are computer readable storage media. In this embodiment, memory 502 includes random-access memory (RAM). In general, memory 502 can include any suitable volatile or non-volatile computer readable storage media. Cache 503 is a fast memory that enhances the performance of processor(s) 501 by holding recently accessed data, and data near recently accessed data, from memory 502.

Program instructions and data used to practice embodiments of the present invention may be stored in persistent storage 505 and in memory 502 for execution by one or more of the respective processor(s) 501 via cache 503. In an embodiment, persistent storage 505 includes a magnetic hard disk drive. Alternatively, or in addition to a magnetic hard disk drive, persistent storage 505 can include a solid-state hard drive, a semiconductor storage device, a read-only memory (ROM), an erasable programmable read-only memory (EPROM), a flash memory, or any other computer readable storage media that is capable of storing program instructions or digital information.

The media used by persistent storage 505 may also be removable. For example, a removable hard drive may be used for persistent storage 505. Other examples include optical and magnetic disks, thumb drives, and smart cards that are inserted into a drive for transfer onto another computer readable storage medium that is also part of persistent storage 505. Software and data 512 are stored in persistent storage 505 for access and/or execution by one or more of the respective processor(s) 501 via cache 503 and one or more memories of memory 502. With respect to corpus of information 110, software and data 512 includes biological member data 112, phenotype information 113, genetic sequence data 114, contig library 115, other sequence data 116, response information 117, and other programs and data (not shown). With respect to system 120, software and data 512 includes k-mer generation program 122, test generation program 124, member data 125, In-group members 126, Out-group members 127, hash tables 128, contig sequences 129, biological analysis program 200, member grouping program 300, cover set generation program 400, and other programs and data (not shown). With respect to user device 141, software and data 512 includes other programs and data (not shown).

Communications unit 507, in these examples, provides for communications with other data processing systems or devices, including resources of corpus of information 110, system 120, and user device 141. In these examples, communications unit 507 includes one or more network interface cards. Communications unit 507 may provide communications, through the use of either or both physical and wireless communications links. Program instructions and data used to practice embodiments of the present invention may be downloaded to persistent storage 505 through communications unit 507.

I/O interface(s) 506 allows for input and output of data with other devices that may be connected to each computer system. For example, I/O interface(s) 506 may provide a connection to external device(s) 508, such as a keyboard, a keypad, a touch screen, and/or some other suitable input device. External device(s) 508 can also include portable computer readable storage media, such as, for example, thumb drives, portable optical or magnetic disks, and memory cards. Software and data used to practice embodiments of the present invention can be stored on such portable computer readable storage media and can be loaded onto persistent storage 505 via I/O interface(s) 506. I/O interface(s) 506 also connect to display 509.

Display 509 provides a mechanism to display data to a user and may be, for example, a computer monitor. Display 509 can also function as a touch screen, such as the display of a tablet computer or a smartphone. Alternatively, display 509 displays information to a user based on a projection technology, such as virtual retinal display, a virtual display, or image projector.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random-access memory (RAM), a read-only memory (ROM), an erasable programmable read-only memory (EPROM or Flash memory), a static random-access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method comprising:
receiving, by one or more computer processors, a request to generate a cover set of k-mers used to detect a first group of biological members, wherein the first group of biological members are related via a taxonomic lineage;
obtaining, by one or more computer processors, a plurality of biological sequence data corresponding to biological members of the first group of biological members from one or more databases;
determining, by one or more computer processors, a set of k-mers respectively associated with a biological member included within the first group of biological members; and
determining, by one or more computer processors, the cover set of k-mers utilized to detect the biological members of the first group by selecting a subset of k-mers from among a superset of k-mers associated with the first group of biological members based on preventing false-positive detections of biological members different from the first group of biological members.

2. The computer-implemented method of claim 1, wherein each biological member is associated with (i) a unique identifier and (ii) a taxonomic lineage.

3. The computer-implemented method of claim 1, wherein receiving the request to generate the cover set of k-mers used to detect the first group of biological members further includes a sensitivity dictate and a second group of biological members to exclude from detection.

4. The computer-implemented method of claim 1, wherein determining the cover set of k-mers utilized to detect the biological members of the first group of biological members is based on a first dictate to determine the cover set of k-mers that includes a minimum number of k-mers required to achieve a dictated detection sensitivity with respect to the first group of biological members.

5. The computer-implemented method of claim 1, further comprising:
converting, by one or more computer processors, biological sequence data corresponding to a first biological member into a binary format; and
performing, by one or more computer processors, a sliding window extraction to identify a plurality of k-mers within the biological sequence data corresponding to the first biological member.

6. The computer-implemented method of claim 5, further comprising:
determining, by one or more computer processors, a hash value corresponding to binary representation of a k-mer, wherein the hash value represents an identifier (ID) corresponding to the k-mer;
generating, by one or more computer processors, one or more key-value tables, wherein a key attribute corresponds to the hash value representing the ID of the k-mer, and value corresponds to a number of occurrences of the k-mer within the biological sequence data corresponding to the first biological member.

7. The computer-implemented method of claim 1, wherein selecting the subset of k-mers from among the superset of k-mers associated with the first group of biological members based on preventing the false-positive detections of biological members different from the first group of biological members further comprises:
identifying, by one or more computer processors, within the received request, a second group of biological members to exclude from detection;
obtaining, by one or more computer processors, a second plurality of biological sequence data corresponding to biological members of the second group of biological members from the one or more databases;
determining, by one or more computer processors, a second superset of k-mers associated the second group of biological members; and
determining, by one or more computer processors, the subset of k-mers based on a relative complement of the superset of k-mers associated with the first group of biological members with respect to the superset of k-mers associated the second group of biological members.

8. The computer-implemented method of claim 7, further comprising:
generating, by one or more computer processors, an origin vector corresponding to each k-mer of the determined set of k-mers included among the first group of biological members, wherein the origin vector is a list of unique identifiers of the biological members that include at least one occurrence of the k-mer; and
removing, by one or more computer processors, one or more origin vectors based on determining that the hash value corresponding to the k-mer of the origin vector is identified among hash values corresponding to the superset of k-mers associated with the second group of biological members.

9. The computer-implemented method of claim 8, further comprising:
performing, by one or more computer processors, a union operation on the remaining origin vectors corresponding to determined set of k-mers included among the first group of biological members to determine a universal set of k-mers associated with the first group of biological members; and
determining, by one or more computer processors, one or more subsets of k-mers from among the universal set of k-mers that includes a quantity of biological members of the first group based on a sensitivity dictate.

10. The computer-implemented method of claim 1, further comprising:
transmitting, by one or more computer processors, a first cover set of k-mers to a user;
in response, receiving, by one or more computer processors, one or more redundancy dictates from the user;
determining, by one or more computer processors, to generate a dictated number other differing cover sets of k-mers capable of identifying the first group of biological members; and
determining, by one or more computer processors, a second cover set of k-mers to identify the biological members of the first group, wherein the second cover set of k-mers includes at least one k-mer common to two or more other differing cover sets of k-mer capable of identifying the biological members of the first group.

11. The computer-implemented method of claim 1, wherein the cover set is utilized to detect whether one or more biological members included among the first group of biological members occurs within a physical sample utilizing a method selected from the group consisting of an in silico analysis of biological sequence data derived from the physical sample and analyzing results of a physical test exposed to the physical sample, wherein the physical test includes an assay array to detect occurrence of k-mers included within the cover set.

12. The computer-implemented method of claim 1, wherein the biological member is selected from the group consisting of: a microorganism, a virus, a protein, a multi-cellular organism, and a particular type of cell associated with the multi-cellular organism.

13. The computer-implemented method of claim 1, wherein the biological sequence includes a selection from the group consisting of: a genome, a gene, a deoxyribonucleic acid (DNA) sequence, a ribonucleic acid (RNA) sequence, a messenger-RNA, an amino acid of a protein, a transcription, and a contig within a genome.

14. A computer program product comprising:
one or more computer readable storage media, and program instructions collectively stored on the one or more computer readable storage media, the program instructions comprising:
program instructions to receive a request to generate a cover set of k-mers used to detect a first group of biological members, wherein the first group of biological members are related via a taxonomic lineage;
program instructions to obtain a plurality of biological sequence data corresponding to biological members of the first group of biological members from one or more databases;
program instructions to determine a set of k-mers respectively associated with a biological member included within the first group of biological members; and
program instructions to determine the cover set of k-mers utilized to detect the biological members of the first group by selecting a subset of k-mers from among a superset of k-mers associated with the first group of biological members based on preventing false-positive detections of biological members different from the first group of biological members.

15. The computer program product of claim 14, wherein each biological member is associated with (i) a unique identifier and (ii) a taxonomic lineage.

16. The computer program product of claim 14, wherein program instructions to receiving the request to generate the cover set of k-mers used to detect the first group of biological members further include a sensitivity dictate and a second group of biological members to exclude from detection.

17. The computer program product of claim 14, wherein program instructions to determine the cover set of k-mers utilized to detect the biological members of the first group of biological members is based on a first dictate to determine the cover set of k-mers that includes a minimum number of k-mers required to achieve a dictated detection sensitivity with respect to the first group of biological members.

18. The computer program product of claim 14, further comprising:
program instructions, collectively stored on the one or more computer readable storage media, to convert biological sequence data corresponding to a first biological member into a binary format; and
program instructions, collectively stored on the one or more computer readable storage media, to perform a sliding window extraction to identify a plurality of k-mers within the biological sequence data corresponding to the first biological member.

19. The computer program product of claim 18, further comprising:
program instructions, collectively stored on the one or more computer readable storage media, to determine a hash value corresponding to binary representation of a k-mer, wherein the hash value represents an identifier (ID) corresponding to the k-mer;
program instructions, collectively stored on the one or more computer readable storage media, to generate one or more key-value tables, wherein a key attribute corresponds to the hash value representing the ID of the k-mer, and value corresponds to a number of occurrences of the k-mer within the biological sequence data corresponding to the first biological member.

20. A computer system comprising:
one or more computer processors, one or more computer readable storage media, and program instructions collectively stored on the one or more computer readable storage media for execution by at least one of the one or more computer processors, the program instructions comprising:
program instructions to receive a request to generate a cover set of k-mers used to detect a first group of biological members, wherein the first group of biological members are related via a taxonomic lineage;
program instructions to obtain a plurality of biological sequence data corresponding to biological members of the first group of biological members from one or more databases;
program instructions to determine a set of k-mers respectively associated with a biological member included within the first group of biological members; and
program instructions to determine the cover set of k-mers utilized to detect the biological members of the first group by selecting a subset of k-mers from among a superset of k-mers associated with the first group of biological members based on preventing false-positive detections of biological members different from the first group of biological members.
